(19) United States
(12) Plant Patent Application Publication
(10) Pub. No.: US 2020/0413585 P1
(43) Pub. Date: Dec. 31, 2020

(54) Raspberry Plant Named 'R15-21-5'

(50) ;

(71) Applicant: Fresas Nuevos Materiales S.A., Huelva (ES)

(72) Inventor: Antonio Refoyo Piriz, Huelva (ES)

(73) Assignee: Fresas Nuevos Materiales S.A., Huelva (ES)

(21) Appl. No.: 16/501,887

(22) Filed: Jun. 28, 2019

(51) Int. Cl. A01H 6/74

(52) U.S. Cl. PLT/204

(57) ABSTRACT

The present invention provides a new and distinct raspberry plant designated as ‘R15-21-5’. The new raspberry cultivar is distinguished from other cultivars by its combination of traits which include very early production on primocanes, branching habit, large light red colored fruit, trapezoidal shaped and fruits suitable for fresh consumption.

Latin name of the genus and species: Rubus idaeus L.

Varietal denomination: ‘R15-21-5’.

### CROSS REFERENCE TO RELATED APPLICATIONS

This application relates to European Plant Breeders' Rights Application No. 201701980, filed on Mar. 01, 2018,. which is herein entirely incorporated by reference for all purposes.

### BACKGROUND OF THE INVENTION

The present invention relates to a new and distinct primocane raspberry variety designated as ‘R15-21-5’.

‘R15-21-5’ was obtained from seed produced by bees pollination of seedlings from Universidad Católica de Chile, according to a collaboration agreement dated Feb. 20, 2014 and was first fruited in Gibraleón (Huelva, Spain) growing fields in 2015. From the seedling and during testing, the plant was designated ‘R15-21-5’.

This new variety was asexually propagated by root cuttings in a controlled environment at Gibraleón, Huelva, Spain. Asexual propagules from the original source have been tested in Cartaya, Moguer and Palos de la Frontera (Huelva, Spain) growing fields and to a limited extent. The properties of this variety were found to be transmissible by such asexual reproduction. This cultivar is stable and reproduces true to type in successive generations of asexual reproduction.

### SUMMARY OF THE INVENTION

This invention concerns a new and distinct cultivar of primocane raspberry variety. The varietal denomination of the new variety is ‘R15-21-5’. The new raspberry cultivar ‘R15-21-5’ is distinguished from other cultivars by its combination of traits which include very high production from October to January on primocanes and from April to June on floricanes; semi-upright branched plant; the vigour of the plant is medium-high, the fruit color is light red, trapezoidal-shaped, firm fruit and large size. The cultivar shows a very early cropping season production in protected culture.

### BRIEF DESCRIPTION OF THE DRAWING

The accompanying colour photographs and traits show typical specimens of the new cultivar at various stages of development as nearly true as it is possible to make in colour reproductions. The pictures were taken during 2018 in Huelva, Spain.

FIG. 1 shows plant habit.

FIG. 2 shows current season's cane.

FIG. 3 shows the upperside leaves.

FIG. 4 shows the downside leaves.

FIG. 5 shows the upperside flowers.

FIG. 6 shows the downside flowers.

FIG. 7 shows the whole fruit on plant.

FIG. 8 shows the fruit in longitudinal cross-section.

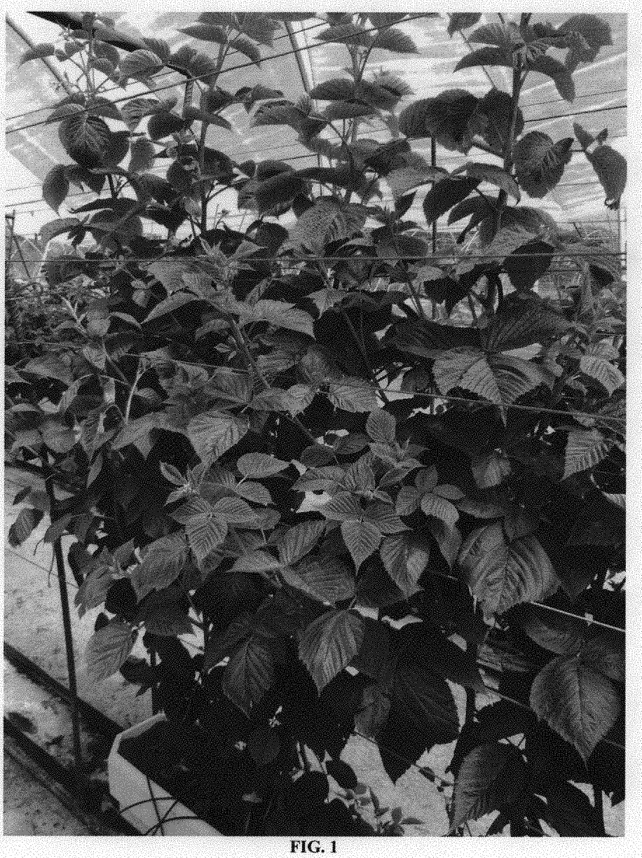

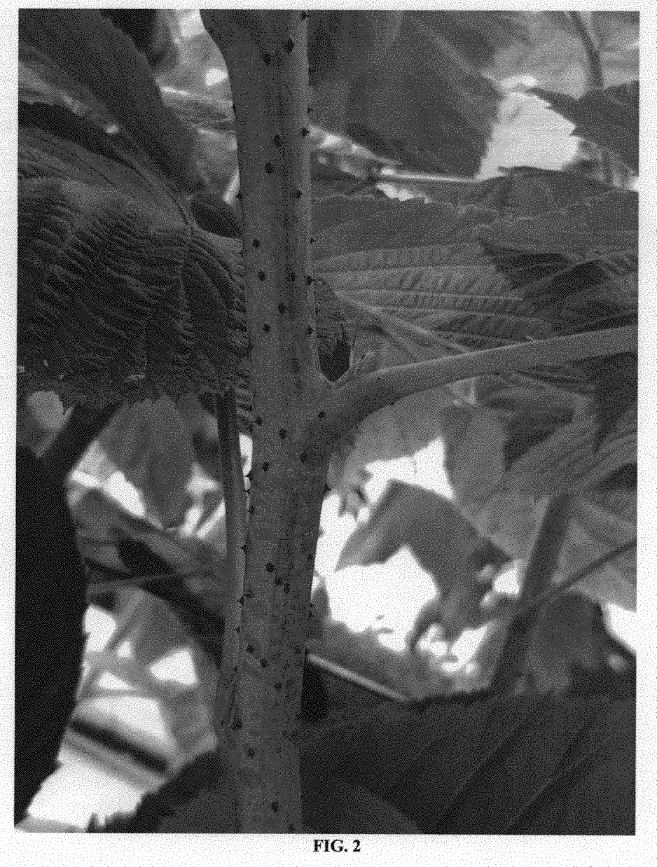

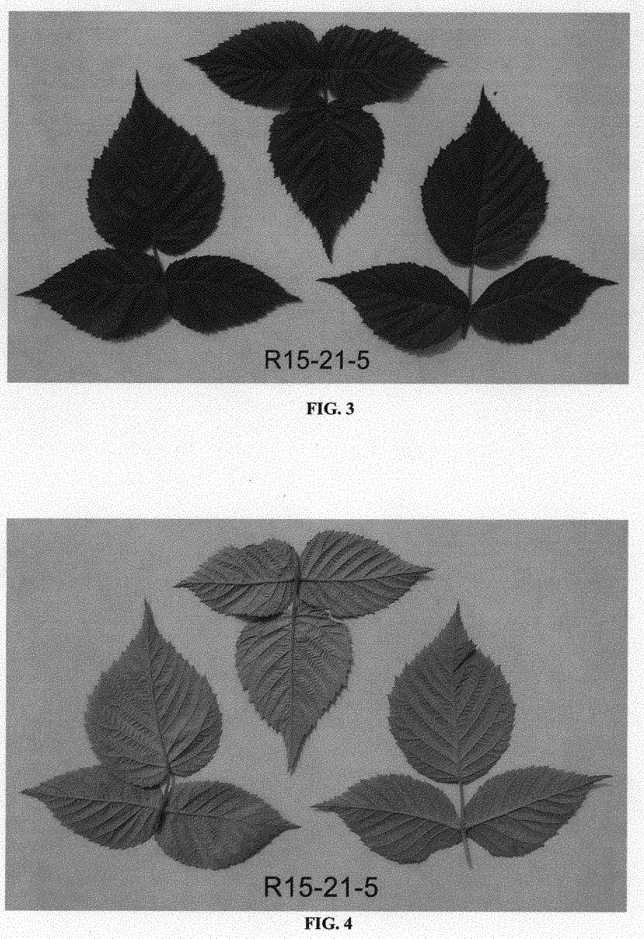

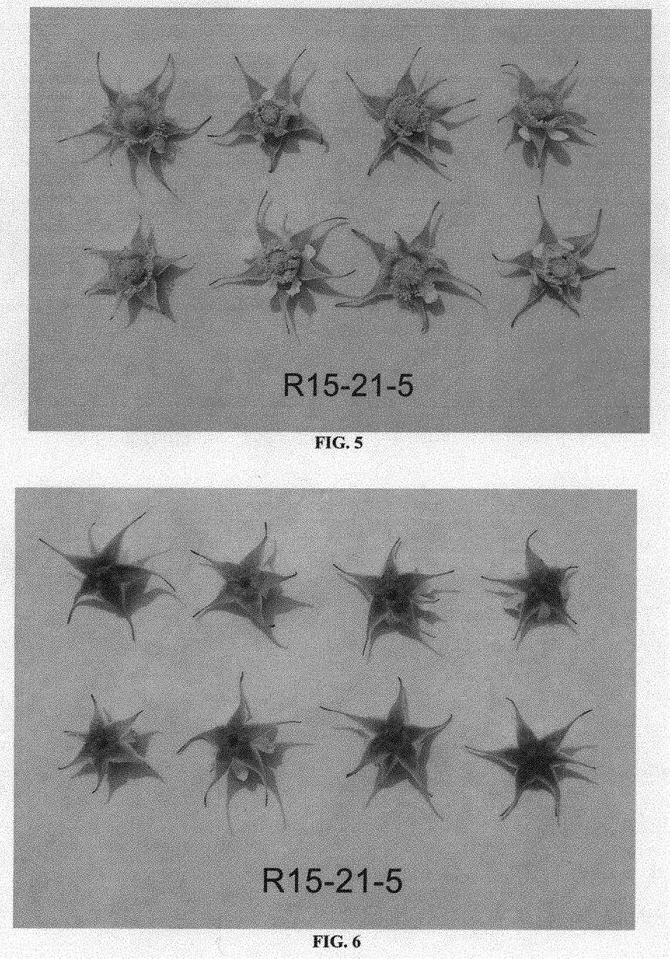

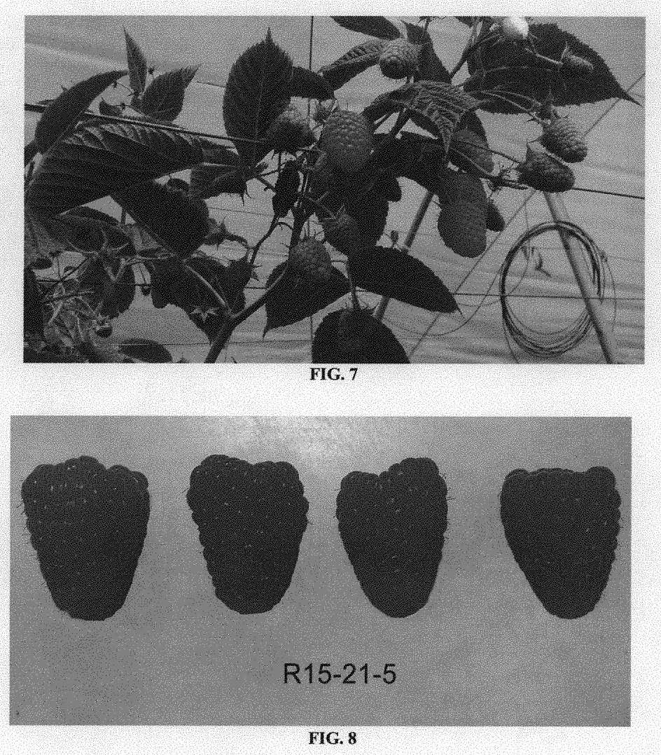

### DETAILED DESCRIPTION OF THE INVENTION

This invention relates to a new and distinctive primocane raspberry cultivar named ‘R15-21-5’. This cultivars is primarily adapted to the climate and growing conditions of the South of Spain (Huelva); Nevertheless considering that is a primocane cultivar, it can be planted in any place of the world that provides the necessary temperatures, this happen in some places of United States as the central coast of California where the nearby Pacific Ocean provides the needed humidity and moderate day temperatures.

‘R15-21-5’ is a semi-upright, medium-high vigorous plant with some branches that produces large fruit size, light red colored and trapezoidal shape.

Fruits are suitable for fresh consumption with excellent quality and high firmness. Fruit production season is much earlier than other cultivars when is grown in protected culture, this allows large quantities of fruits in winter and early spring. Fruit is easily removed from its receptacle, with adequate width of the cavity, and easily visible on branches.

‘R15-21-5’ have not been noted to be sensitive to any pest in Huelva (Spain). Specifically, it's relatively tolerant to aphids and spider mites.

Plants of ‘R15-21-5’ are slightly susceptible to powdery mildew (Podosphaera aphanis) when greenhouse grown.

‘R15-21-5’ can be asexually propagated by root suckers, root cuttings and other know methods of propagating raspberry plants. No off-type plants have been observed in the history of asexual propagation of this cultivar by either method.

The results showed below describe production, weight per fruit and Brix degrees of the variety ‘R15-21-5’ from October 2016 to January 2017 on primocanes (Table 1) and from April 2017 to June 2017 on floricanes (Table 2). The results come from three trials located in Gibraleón, Palos de la Frontera and Bonares (Huelva, Spain). Each trial was a randomized complete block design with three repetitions and 6 plants per plot. Root cuttings was planting on Jul. 17, 2016 and was cultivated until May 31, 2017. It was cultivated under standard conditions of raspberry cultivation in Huelva, Spain.

TABLE 1

| Variety | Location | Cultivation method | Yield | brix | weight |
|---|---|---|---|---|---|
| R15-21-5 | Gibraleón | Hydroponic | 1699 | 9.5 | 6.6 |
| R15-21-5 | Palos Ftra | Hydroponic | 1826 | 8.0 | 6.3 |
| R15-21-5 | Bonares | Raised bed | 1518 | 8.6 | 5.7 |

Table 1. fruit production, weight per fruit and Brix degrees of the variety ‘R15-21-5’ from October 2016 to January 2017 on primocanes. Each value is the result of the average of three repetitions of 6 plants, yield, total commercial yield in grams per plant; weight, weight in grams per first quality fruits; brix, Brix degrees.

TABLE 2

| Variety | Location | Cultivation method | Yield | brix | weight |
|---|---|---|---|---|---|
| R15-21-5 | Gibraleón | Hydroponic | 1317 | 9.3 | 7.5 |
| R15-21-5 | Palos Ftra | Hydroponic | 1665 | 8.1 | 7.1 |
| R15-21-5 | Bonares | Raised bed | 1021 | 7.6 | 6.3 |

Table 2. fruit production, weight per fruit and Brix degrees of the variety ‘R15-21-5’ from April 2017 to June 2017 on floricanes. Each value is the result of the average of three repetitions of 6 plants. yield, total commercial yield in grams per plant; weight, weight in grams per first quality fruits; brix, Brix degrees.

The following traits in combination distinguish raspberry variety ‘R15-21-5’ from known raspberry varieties. It is based in UPOV terminology and color references are made to The Royal Horticultural Society Colour Chart, 2015 Edition, except where general terms of ordinary dictionary significance are used.

Following observations describe primocane variety with fruit production from October 2016 to January 2017 on primocanes and from end of April 2017 to June 2017 on floricanes, in 30-liter container in a greenhouse located in Huelva (Spain). Root cuttings was used for planting in July 2016.

### General

| Characteristics | R15-21-5 |
|---|---|
| Main bearing type | Both, on previous year's cane |
| | and on current year's cane |
| Time of cane emergence | Early |
| Time of beginning of flowering | Very early |
| on current season's cane | |
| Time of beginning of fruit | Very early |
| ripening on current year's cane | |
| Length of fruiting period on | Long |
| current year's cane | |

### Root

| Characteristics | R15-21-5 |
|---|---|
| Habit | Dense |
| Thickness | Medium to thin |
| Color | RHS, 164A-164B |

### Plant

| Characteristics | R15-21-5 |
|---|---|
| Habit | Semi-upright |
| Vigor | Medium to high |
| Number of current | Many |
| season's canes | |

Very young shoot

| Characteristics | R15-21-5 |
|---|---|
| Anthocyanin coloration of | Absent |
| apex during rapid growth | |
| Color | RHS, 144B-144C |

Observations made when the shoots are about 15 cm long
Current season's cane

| Characteristics | R15-21-5 |
|---|---|
| Bloom | Medium |
| Anthocyanin coloration | Absent or very weak |
| Length of internode | Medium, 5.0 to 7.0 cm |
| Length of vegetative bud | Medium, 0.5 to 1.0 cm |
| Length | Long, 190 to 200 cm |
| Diameter | 1.28 to 1.36 cm |
| Color | RHS, 143B-144A |

Observations made just before harvest, made in the middle third of the current season's cane, when the cane is about 1 m to 1.50 m long.
Lateral branch

| Characteristics | R15-21-5 |
|---|---|
| Habit | Upright |
| Bloom | Medium |
| Anthocyanin coloration | Absent |
| Length of internode | Medium, 5.0 to 7.0 cm |
| Length | Long, 68 to 110 cm |
| Diameter | 0.68 to 0.87 cm |
| Color | RHS, 144B-144C |

Observations made just before harvest, made in the middle third of the current season's cane, when the cane is about 1 m to 1.50 m long.

### Spines

| Characteristics | R15-21-5 |
|---|---|
| Presence | Present |
| Density | Sparse, 4.39/cm² |
| Size of base | Small, 0.10 to 0.20 cm |
| Length | Short, 0.20 to 0.30 cm |
| Shape | Deltoid |
| Color | RHS, 184A |

Observations on spines made in the middle third of the current season's cane, when the cane is about 1 m to 1.50 m long.

### Petiole

| Characteristics | R15-21-5 |
|---|---|
| Spines density | Very few, 5 to 8 |
| Length | 5.5 to 8.5 cm |
| Color | RHS, 144B-144C |
| Diameter | 0.30 to 0.37 cm |

Observations made on fully developed leaves from the middle third of the cane.

### Stipule

| Characteristics | R15-21-5 |
|---|---|
| Number per leaf | 2 |
| Length | 0.5 to 0.8 cm |
| Width | <0.1 cm |
| Shape | Upright |
| Color | RHS, 144B |

Observations made on fully developed leaves from the middle third of the cane.

### Leaf

| Characteristics | R15-21-5 |
|---|---|
| Green color of upper side | RHS, NN137B-NN137C |
| Predominant number of leaflets | 3 |
| Profile of leaflets in cross section | Concave |
| Rugosity | Medium |
| Relative position of lateral leaflets | Touching |
| Length | Long, 26.5 to 28.0 cm |
| Width | Broad, 28.0 to 30.5 cm |
| Shape | Unipinnate, Imparipinhate |
| Shape of tip | Acuminate |
| Shape of base | Rounded |
| Shape of margin | Serrate |
| Length of terminal leaflet | Long, 15.5 to 18.5 cm |
| Width of terminal leaflet | Broad, 14.5 to 16.5 cm |
| Length of lateral leaflet | Long, 14.5 to 15.3 cm |
| Width of lateral leaflet | Broad, 9.2 to 10.0 cm |

Observations made on fully developed leaves from the middle third of the cane.

### Pedicel

| Characteristics | R15-21-5 |
|---|---|
| Number of spines on pedicel | Medium, 15 to 30 |
| Diameter | 0.11 to 0.13 cm |
| Color | RHS, 143C-144B |

Observations made during the autumn fruiting period

### Peduncles

| Characteristic | R15-21-5 |
|---|---|
| Presence of | Absent |
| anthocyanin coloration | |
| Diameter | 0.18 to 0.25 cm |
| Color | RHS, 143C-144B |

Observations made during the autumn fruiting period

### Petal

| Characteristics | R15-21-5 |
|---|---|
| Number per flower | 5 to 7 |
| Length | 0.5 to 0.6 cm |
| Width | 0.3 to 0.4 cm |
| Shape | Elliptic |
| Apex shape | Obcordate |
| Base shape | Acute |
| Margin shape | Regular |
| Color | RHS, NN155A-NN155B |

Observations made during the autumn fruiting period

### Sepal

| Characteristics | R15-21-5 |
|---|---|
| N° per flower | 5 |
| Length | 1.12 to 1.20 cm |
| Width | 0.5 to 0.7 cm |
| Shape | Deltoid |
| Apex shape | Aristate |
| Margin shape | Regular |
| Color | RHS, 143B-143C |

Observations made during the autumn fruiting period

### Flower

| Characteristics | R15-21-5 |
|---|---|
| Size | Large |
| Diameter | 1.9 to 2.7 cm |

Observations made during the autumn fruiting period
Reproductive organs

| Characteristics | R15-21-5 |
|---|---|
| Number of pistils/flower | 126 to 142 |
| Pistil length | 0.3 to 0.5 cm |
| Ovary shape | Oval |
| Ovary color | RHS, 144D |
| Style length | 0.3 to 0.4 cm |
| Style color | RHS, 155A-155B |
| Number of stamens/flower | 102 to 116 |
| Stamen length | 0.4 to 0.6 cm |
| Stamen shape | Oval |
| Stamen color | RHS, 155A-155B |
| Pollen color | RHS, 155B |

Observations made during the autumn fruiting period

### Fruit

| Characteristics | R15-21-5 |
|---|---|
| Length | Long, 2.6 to 3.0 cm |
| Width | Broad, 2.5 to 2.8 cm |
| Ratio length/width | Large |
| General shape | Trapezoidal |
| in lateral view | |
| Size of single drupe | Large, 0.3 to 0.4 cm |
| Color | RHS, 46B to 46C |
| Glossiness | Strong |
| Firmness | Firm |
| Adherence to plug | Very weak |
| Number of drupelets | 124 to 148 |
| Diameter hollow center | 0.9 to 1.1 cm |

Observations made on fruit picked during the second and third harvest of the autumn fruiting period.

The invention claimed is:

1. A new and distinct cultivar of raspberry plant named ‘R15-21-5’ as described and shown herein.
